(19) United States
(12) Patent Application Publication
(10) Pub. No.: US 2023/0330480 A1
(43) Pub. Date: Oct. 19, 2023

(54) TONGUE EXERCISE APPARATUS FOR SNORING PREVENTION

(71) Applicant: LTPRO. CO. KR, Gyeonggi-do (KR)

(72) Inventor: Yoon Hwoa KO, Gyeonggi-do (KR)

(21) Appl. No.: 18/024,276

(22) PCT Filed: Sep. 29, 2021
(86) PCT No.: PCT/KR2021/013409

§ 371 Date: Mar. 2, 2023

(30) Foreign Application Priority Data
Sep. 29, 2020 (KR) 10-2020-0126679

(51) Int. Cl. A63B 23/03; A63B 23/18; A61B 17/24

(52) U.S. Cl. CPC A63B 23/032; A63B 23/18; A61B 17/244; A63B 2220/17; A63B 2220/833

(57) ABSTRACT

The purpose of the present disclosure is to provide a tongue exercise apparatus for snoring prevention, wherein the apparatus can be useful for exercising the tongue in order to prevent snoring. A tongue exercise apparatus according to the present disclosure is a tongue exercise apparatus that a user can hold by hand and use to perform tongue exercises, the apparatus comprising: a handle which the user can hold by hand; a tooth engagement part which extends from the end of the handle and has an engagement groove that can engage with the ends of the teeth of the user; and an exercise assistant part which is connected to the tooth engagement part so as to be able to enter the mouth of the user, and has a protrusion portion that protrudes, in a direction intersecting with the direction in which the tooth engagement part extends from the handle, so that the tongue of the user can contact the protrusion.

### TECHNICAL FIELD

The present disclosure relates to a tongue exercise apparatus for snoring prevention, and more particularly, a tongue exercise apparatus for snoring prevention which can be used to train tongue muscles by the user's simply holding it with a hand and inserting its part into an inside of a mouth in order to prevent snoring.

### BACKGROUND

Snoring adversely affects health. In particular, many people who snore are accompanied by sleep apnea, in which people stop breathing for a while. Because being unable to breath properly due to the snoring, the amount of oxygen which human body needs is lacking. Because of this, there are reports that brain pressure (cerebral pressure), blood pressure, and central venous pressure increase, and the risk of complications such as hypertension, arrhythmia, myocardial infarction, stroke, dementia, diabetes, and glaucoma may increase.

It has been reported that only about 15% of people need surgery because of the snoring. This is because about 15% of the snoring is caused by problems with the uvula or nose that require surgery. If the uvula blocks the airway and causes the snoring, the snoring can be prevented by removing the sagging uvula or by performing surgery to tighten it. However, since the snoring is caused not only by the uvula but also by complex causes such as the tongue, obesity, and facial structure, the surgery of removing only the uvula tissue cannot fundamentally solve it.

The snoring is most often caused by the tongue blocking the airway. When lying down while sleeping, the tongue is pushed toward the airway by gravity, thereby narrowing the airway and causing the snoring. Weak tongue muscles increase the risk of snoring. The tongue muscles weaken with age, so snoring is more common in older people.

Recently, research results have been announced that only tongue and mouth exercises can alleviate the snoring. The reason why snoring symptoms improve after exercising the tongue and mouth is because the tissue in the area that causes snoring is strengthened.

To effectively prevent snoring, regular and steady exercise is necessary. However, there is currently no practical device capable of assisting tongue exercise to prevent the snoring.

### SUMMARY

### Technical Problem

The present disclosure has been made in view of the above points, and the purpose of the present disclosure is to provide a tongue exercise apparatus for preventing snoring that can be usefully used for tongue exercise to prevent snoring.

### Solution to Problem

In order to solve the purpose as described abovel, a tongue exercise apparatus which a user is able to hold with a hand and use to exercise a tongue according to the present disclosure comprises: a handle which the user is able to hold with the hand; a teeth engagement portion extended from an end portion of the handle, and having one or more engagement grooves configured to be engageable with an end portion of teeth of the user; and an exercise assistant portion connected to the teeth engagement portion to be insertable into an inside of a mouth of the user, and having a protrusion portion protruding in a direction of crossing with a direction in which the teeth engagement portion is extended from the handle to be contactable with the tongue of the user.

A plurality of the engagement grooves may be disposed to be spaced apart from each other along the direction in which the teeth engagement portion is extended from the handle.

The teeth engagement portion is made of a circular cylinder shape, and the one or more engagement grooves may be provided in an annular shape on an outer circumferential surface of the teeth engagement portion.

The exercise assistant portion may be made of silicon material.

A reinforcing material or stiffener configured to be elastically deformable may be disposed inside the exercise assistant portion.

The teeth engagement portion and the exercise assistant portion may be made of silicon material and may be formed integrally with each other.

The teeth engagement portion may be replaceably coupled to the handle.

The tongue exercise apparatus according to the present disclosure may further comprise a support body disposed at an end of the teeth engagement portion to support the exercise assistant portion.

The exercise assistant portion may be replaceably coupled to the support body.

The exercise assistant portion may be rotatably coupled to the support body.

The exercise assistant portion may comprise: a first wing body protruded from one side of the protrusion portion; and a second wing body protruded from an other side of the protrusion portion.

The support body may comprise a contact curved surface portion formed in a curved surface shape to be capable of contact a roof of the mouth of the user.

A tongue exercise apparatus according to the present disclosure may further comprise: a handle chamber disposed inside the handle to store liquid; a teeth engagement portion chamber disposed inside the teeth engagement portion to be connected to the handle chamber; a support body chamber disposed inside the support body to be connected to the teeth engagement portion chamber; an insertion groove formed at one side of the support body; a connection hole formed at an inside of the support body to connect the support body chamber and the insertion groove; an opening and closing structure installed at the support body chamber and configured to open and close the connection hole; a spring installed at the insertion groove to apply an elastic force to the protrusion portion in a direction away from the connection hole; an insertion portion protruding at one side of the protrusion portion; an operation protrusion protruding at the insertion portion to be contacted with the opening and closing structure and press the opening and closing structure in a direction of opening the connection hole; and a passage extended from the operation protrusion to an outer surface of the protrusion portion to guide the liquid of the support body chamber to the outer surface of the protrusion portion.

A tongue exercise apparatus according to the present disclosure may further comprise: an air hole formed at the handle to flow outside air into the handle chamber; a plunger movably installed in the air hole to regulate flow of air through the air hole; and a spring installed inside the air hole to apply an elastic force to the plunger in a direction of blocking the flow of the air through the air hole.

The exercise assistant portion comprises: a curved portion provided on one side of the protrusion portion to be contacted with the tongue of the user; and an assistant protrusion portion protruding from a center of an end of the protrusion portion to be contactable with the tongue of the user.

The exercise assistant portion may comprise a scraper protruding from the assistant protrusion portion so that the scraper contacts the tongue of the user to scrape away a foreign substance on the tongue of the user.

The scraper may be formed of an arcuately bent shape so that a center portion of the scraper is positioned deeper in the mouth of the user than both edges of the scraper.

The protrusion portion may have a base surface portion, wherein a plurality of protrusions are protrudingly formed on the base surface portion.

A tongue exercise apparatus according to the present disclosure may further comprise: a counting module configured to count a number of times the protrusion portion is pressed or moved by the tongue of the user.

A tongue exercise apparatus according to the present disclosure may further comprise: a controller configured to receive measurement information from the counting module, and transmit the measurement information to a personal terminal of the user.

A tongue exercise apparatus according to the present disclosure may further comprise: an output module configured to output the measurement information of the counting module.

### Advantageous Effects of Invention

A tongue exercise apparatus according to the present disclosure can assist tongue exercise so that a user can effectively train tongue muscles. Therefore, it is effective in preventing snoring caused by the obstruction of the airway due to the weakening of the tongue muscles.

In addition, a tongue exercise apparatus according to the present disclosure can be used to remove foreign substances such as cover attached to the tongue while exercising the tongue.

Additionally, a tongue exercise apparatus according to the present disclosure has a simple structure, is easy to manage, and can be used by conveniently holding it by the user by hand.

### BRIEF DESCRIPTION OF THE DRAWINGS

FIG. 1 is a perspective view for showing a tongue exercise apparatus for preventing snoring according to an embodiment of the present disclosure.

FIG. 2 is an exploded perspective view for showing the tongue exercise apparatus shown in FIG. 1.

FIG. 3 is a perspective view for showing an exercise assistant part of the tongue exercise apparatus shown in FIG. 1.

FIG. 4 is a side view for showing an exercise assistant part of the tongue exercise apparatus shown in FIG. 1.

FIG. 5 shows an example of use of a tongue exercise apparatus illustrated in FIG. 1.

FIG. 6 shows a modified example of an exercise assistant portion.

FIGS. 7 to 26 show various modified examples of tongue exercise apparatuses.

### DETAILED DESCRIPTION OF EMBODIMENTS

Hereinafter, a tongue exercise apparatus for preventing snoring according to an embodiment of the present disclosure will be described in detail with reference to the drawings.

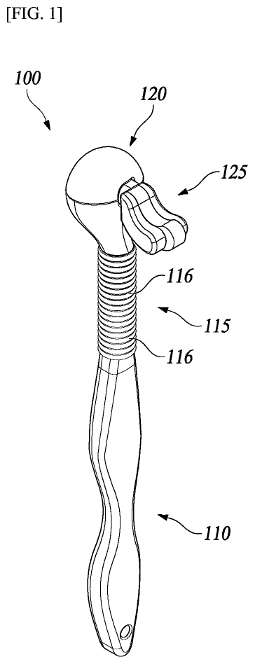

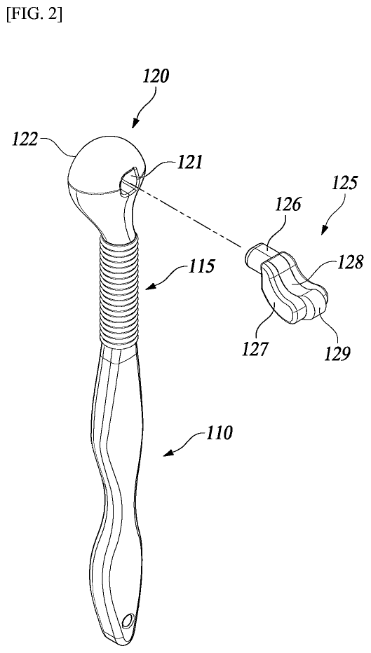

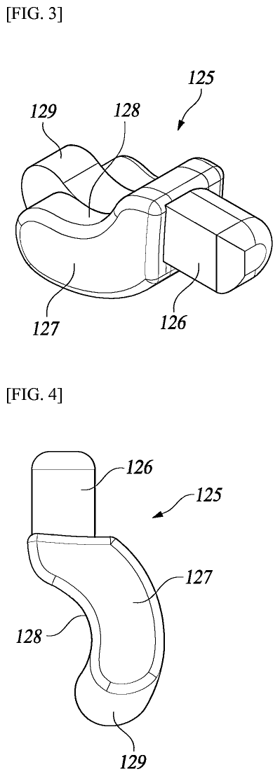

FIG. 1 is a perspective view for showing a tongue exercise apparatus for preventing snoring according to an embodiment of the present disclosure, FIG. 2 is an exploded perspective view for showing the tongue exercise apparatus shown in FIG. 1, and FIG. 3 is a perspective view for showing an exercise assistant part of the tongue exercise apparatus shown in FIG. 1, and FIG. 4 is a side view for showing an exercise assistant part of the tongue exercise apparatus shown in FIG. 1.

As shown in the drawing, a tongue exercise apparatus (100) according to an embodiment of the present disclosure includes a handle (110), a tooth engagement portion (115) connected to the end of the handle (110), a support body (120) connected to the end portion of the tooth engagement portion (115), and an exercise assistant portion (125) coupled to the support body (120) to enter into the user's mouth in order to reach the user's tongue (10). The tongue exercise apparatus (100) can be used to train the tongue muscles by simply holding with the user's hand. That is, by a way in which the user using the tongue exercise apparatus (100) according to an embodiment of the present disclosure puts the tongue (10) on the exercise assistant portion (125) and moves it, the tongue muscles can be effectively trained, and the snoring caused by the tongue muscles which do not have enough strength and is stretched thereby blocking the airway can be prevented.

The handle (110) has a bar shape that a user can hold with one hand. The shape of the handle (110) is not limited to a special shape, and any shape is possible as long as the user can hold it by hand. And the handle (110) may be made of various materials such as plastic or metal.

The tooth engagement portion (115) extends from the end portion of the handle (110). The tooth engagement portion (115) is provided with a plurality of engagement grooves (116) into which the end portions of the user's teeth (20) can be engaged. The plurality of engagement grooves (116) are spaced apart in turn along a direction elongated from the tooth engagement portion (115). The tooth engagement portion (115) has a cylindrical shape, and the engagement groove (116) is provided in an annular shape on the outer circumferential surface of the tooth engagement portion (115).

The user of the tongue exercise apparatus (100) according to an embodiment of the present disclosure inserts the exercise assistant portion (125) into the mouth at an appropriate depth and inserts the end portions of the tooth (20) into the engagement grooves (116) to fix the position of the exercise assistant portion (125) inside the mouth. In addition, in a state in which the exercise assistant portion (125) is fixed in the mouth, the tongue (10) may be put on the exercise assistant portion (125) and be moved to train the tongue muscles.

Because the plurality of engagement grooves (116) are fomred along the length direction of the tooth engagement portion (115), the user can insert the tooth (20) into the appropriate engagement grooves (116) according to the user's oral structure to be fixed at a position in which the exercise assistant portion (125) can be conveniently used. And, the insertion depth of the exercise assistant portion (125) can be adjustable by changing the position of the engagement groove (116) into which the tooth (20) is inserted among the plurality of engagement grooves (116).

The exercise load of the tongue (10) may be changed according to a depth into which the exercise assistant portion (125) is inserted in the mouth. For example, as a distance between the tongue (10) and the exercise assistant portion (125) is farther, the exercise assistant portion (125) can be pushed by moving the tongue (10) less, thereby reducing the exercise load of the tongue (10). On the other hand, as a distance between the tongue (10) and the exercise assistant portion (125) is closer, the exercise assistant portion (125) can be pushed by moving the tongue (10) more, thereby increasing the exercise load of the tongue (10).

In this way, the tongue exercise apparatus (100) according to an embodiment of the present disclosure has a plurality of engagement grooves (116) which the teeth 20 can be engaged with and are spaced apart from each other, so that the user can place and use the exercise assistant portion (125) in a convenient position according to the user's oral structure and can use it by adjusting the exercise load in various ways.

The shape of the tooth engagement portion (115) is not limited to the shapes illustrated in the figures, and may be variously changed. And, the engagement groove (116) may be changed into various shapes in which the end portion of the user's teeth (20) can be inserted or engaged, in addition to a shape like the annular shape illustrated in the figures. In addition, the length of the tooth engagement portion (115), the depth of the engagement groove (116), and the spacing between the engagement grooves (116) can be designed in various ways. In addition, the number of engagement grooves (116) provided in the tooth engagement portion (115) may be changed to one or more of various numbers.

Because the tooth engagement portion (115) is a part contacting the user's tooth (20), it is preferable to be made of a material that does not cause pain to the tooth (20), or does not damage the tooth (20), is not easily deformed even when pressed by the tooth (20), and is not harmful to the human body. For example, the tooth engagement portion (115) may be made of a silicon material. In addition, the tooth engagement portion (115) may be made of any various materials that can satisfy the above conditions.

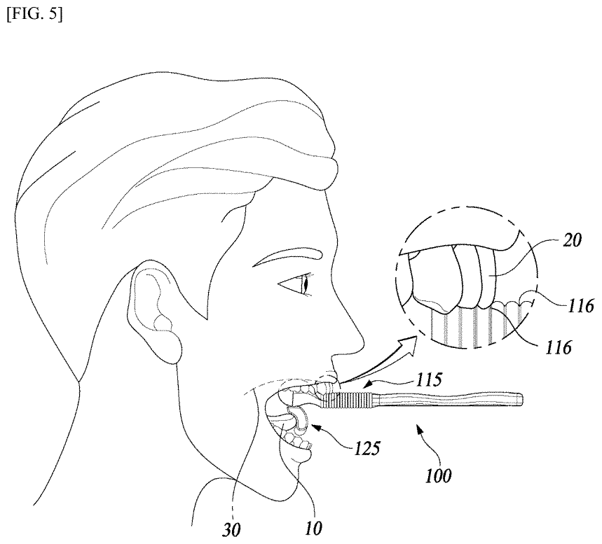

The support body (120) is connected to the end portion of the tooth engagement portion (115) to support the exercise assistant portion (125). The support body (120) may enter into the user's mouth together with the exercise assistant portion (125). An insertion groove 121 into which the exercise assistant portion (125) is inserted is included in the support body (120). In addition, one side of the support body (120) is provided with a contact curved surface portion (122) that can adhere to the roof of the user's mouth (30; see FIG. 5). The contact curved surface portion (122) is formed in a curved shape so as to stably adhere to the roof of the user's mouth (30) and not to cause pain in the roof of the mouth (30). The support body (120) may stably support the exercise assistant portion (125) by closely contacting with the roof of the mouth (30) in the user's mouth.

Since the support body (120) is inserted into the user's mouth and comes into contact with the roof of the mouth (30), it is preferable to be made of a material having elasticity so that it can gently touch the roof of the mouth (30) without causing pain to the user. For example, the support body (120) may be made of a silicon material. The support body (120) may be molded together with the tooth engagement portion (115) in various ways such as injection molding and so on, or may be manufactured separately from the tooth engagement portion (115) and then coupled with the tooth engagement portion (115).

The exercise assistant portion (125) is coupled to the support body (120) to be inserted into the user's mouth. The exercise assistant portion (125) includes an insertion portion (126) inserted into the insertion groove (121) of the support body (120), and a protrusion portion (127) connected to the end of the insertion portion (126). The exercise assistant portion (125) is detachably coupled to the support body (120) in such a way that the insertion portion (126) is inserted into the insertion groove (121).

A protrusion portion (127) is coupled to the support body (120) and protrudes in a direction of crossing the length direction of the tooth engagement portion (115), that is, a direction of crossing a direction in which the tooth engagement portion (115) extends from the handle (110). One side of the protrusion portion (127) is provided with a curved portion (128) which the user's tongue 10 can contact. The curved portion (128) is formed in a gently curved shape from one end of the protrusion portion (127) toward the other end of the protrusion portion (127). The user may stably push the protrusion portion (127) while putting the tongue (10) on the curved portion (128). An assistant protrusion (129) that is contactable with the user's tongue (10) is protrudedly provided at the center of the end of the protrusion portion (127). The user may put the tongue (10) on the assistant protrusion (129) and push the end of the protrusion portion (127) toward an outward direction of the mouth or push the protrusion portion (127) in a left or right direction.

The exercise assistant portion (125) may be made of a material that can be elastically deformed by being pushed by the user's tongue (10). For example, the exercise assistant portion (125) may be made of a silicone material having elasticity. In addition, the exercise assistant portion (125) is made of various materials such as a material that is elastically deformable by being pushed by the tongue (10) or a material that can maintain its shape without being easily deformed so that the user can train the tongue muscles by putting the tongue (10) on the exercise assistant portion (125).

Hereinafter, a method of using the tongue exercise apparatus (100) according to an embodiment of the present disclosure will be described.

As shown in FIG. 5, the user can hold the handle (110) with a hand and open the mouth to put the support body (120) and the exercise assistant portion (125) into the mouth. And, the position of the exercise assistant portion (125) is adjusted so that the exercise assistant portion (125) can reach the tongue (10), and the tooth engagement portion (115) is brought into close contact with the upper teeth (20). At this time, the end of the upper tooth (20) is inserted into the engagement groove (116) of the tooth engagement portion (115) so that the tooth engagement portion (115) is caught on the tooth (20). Accordingly, the exercise assistant portion (125) may be fixed in position in the mouth. In this state, the user can train the tongue muscles by placing the tongue (10) on the protrusion portion (127) and pushing the protrusion portion (127).

For example, by rolling the entire tongue (10) and strongly pushing the protrusion portion (127) forward repeatedly, the central muscles and the left and right side muscles of the tongue (10) can be simultaneously trained. Additionally, by putting the tongue (10) on the left or right side of the protrusion portion (127) and pushing the tongue (10) in the opposite direction repeatedly, the muscles of the left side or right side of the tongue (10) can be trained. Because the protrusion portion (127) is elastically deformed when pressed by the tongue (10) and then restored when the pressing force is released, the user can effectively train the tongue muscles by repeating operations of deforming the protrusion portion (127).

After using the tongue exercise apparatus (100), the exercise assistant portion (125) may be separated from the support body (120), and the exercise assistant portion (125) and the rest part may be washed and stored. The tongue exercise apparatus (100) does not occupy a large storage space and is easy to store because the exercise assistant portion (125) is detachable.

If the exercise assistant portion (125) is damaged during use, the tongue exercise apparatus (100) according to one embodiment of the present disclosure can be used by replacing only the exercise assistant portion (125) with a new one.

The user using the tongue exercise apparatus (100) according to an embodiment of the present disclosure can perform various types of tongue exercises by replacing the exercise assistant portion with another type as needed.

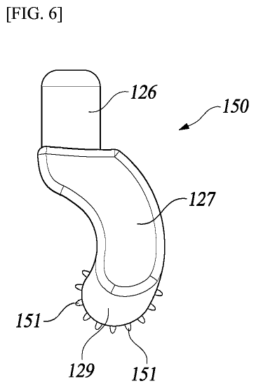

FIG. 6 shows another type of an exercise assistant portion (150).

The exercise assistant portion (150) shown in FIG. 6 includes an insertion portion (126), a protrusion portion (127) connected to the end of the insertion portion (126), an assistant protrusion portion (129) protruding from the center of the end of the protrusion portion (127), and a plurality of scrapers (151) provided at the assistant protrusion portion (129). Here, the insertion portion (126), the protrusion portion (127), and the assistant protrusion portion (129) are as described above.

The plurality of scrapers (151) are disposed to be spaced apart from each other along the outer circumference of the assistant protrusion portion (129). The scraper (151) may come into contact with the user's tongue 10 and scrape foreign substances attached to the user's tongue (10). The scraper (151) may have a plate-like structure having a width corresponding to the width of the assistant protrusion portion (129).

The user using the exercise assistant portion (150) according to the present embodiment may remove foreign substances such as coat stuck on the tongue (10) by putting the upper portion of the tongue (10) on the scraper (151) and reciprocating the upper portion of the tongue (10). Therefore, the tongue (10) can be washed and cleaned, and through this, bad breath can be removed.

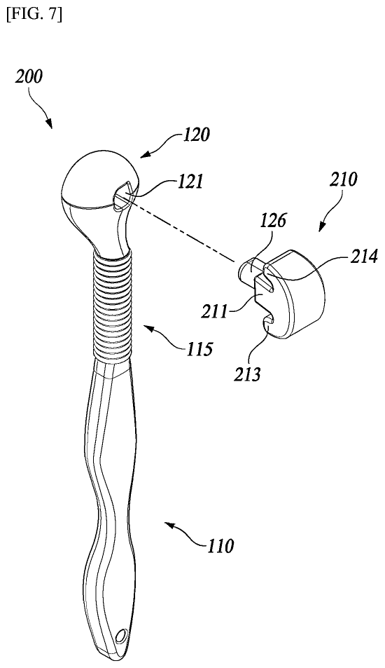

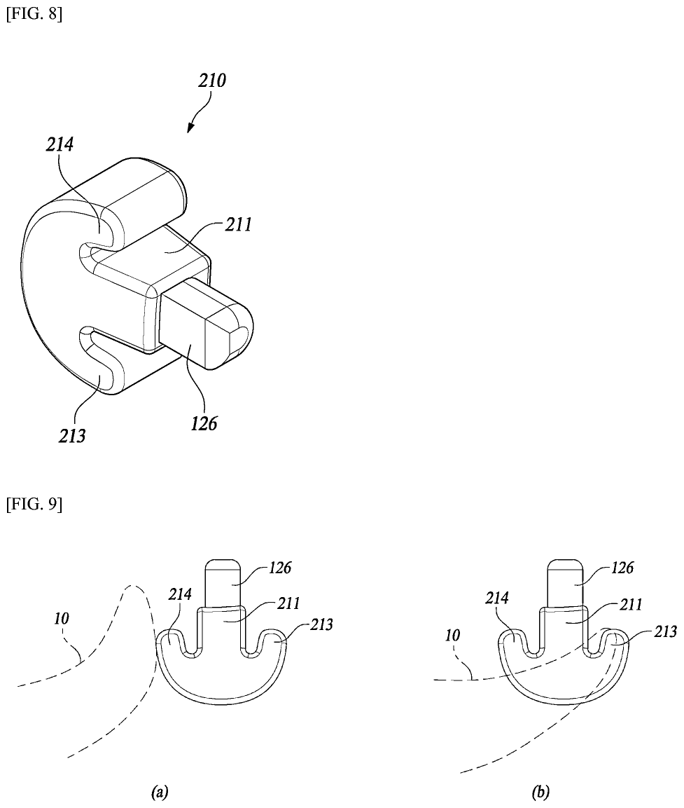

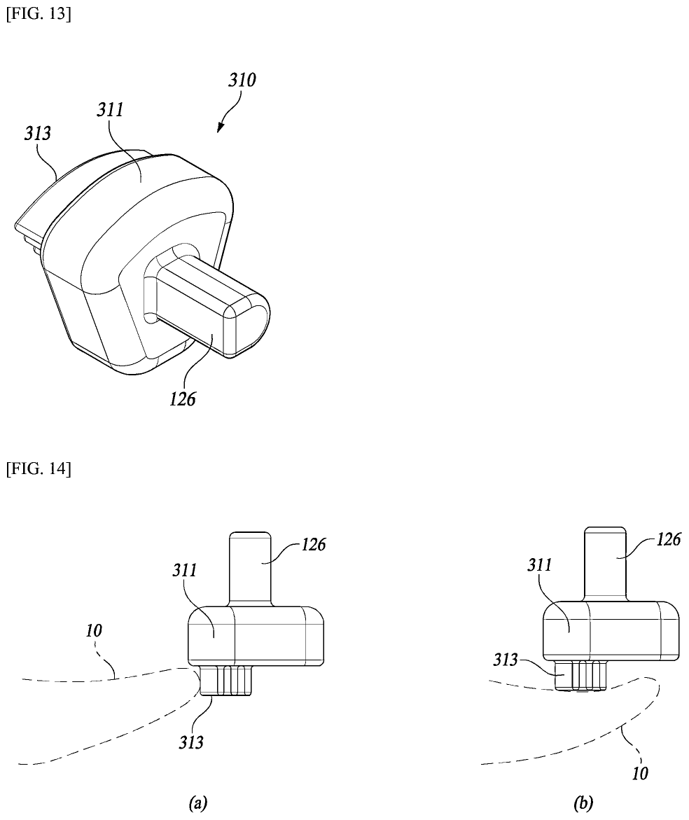

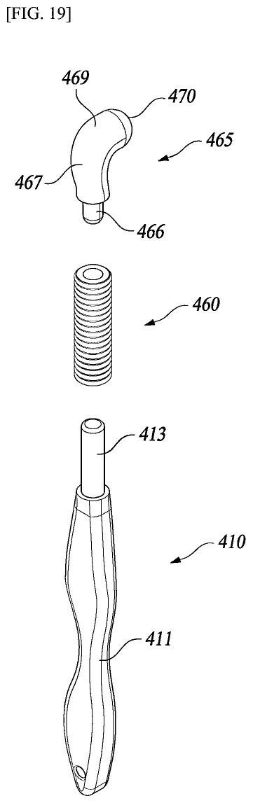

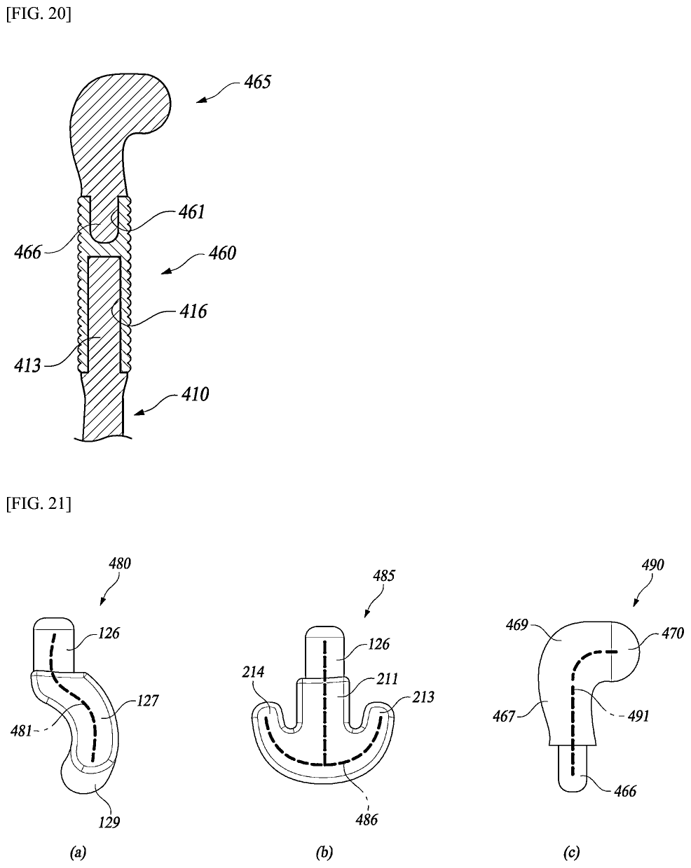

On the other hand, FIGS. 7 to 26 show various modifications of tongue exercise apparatuses.

First, a tongue exercise apparatus (200) shown in FIGS. 7 to 9 includes a handle (110), a tooth engagement portion (115) connected to the end of the handle (110), a support body (120) connected to the end of the tooth engagement portion (115), and an exercise assistant portion (210) coupled to the support body (120). In this tongue exercise apparatus (200), the exercise assistant portion (210) is modified compared to the tongue exercise apparatus (100) described above.

The exercise assistant portion (210) is coupled to the support body (120) so as to be inserted into the user's mouth. The exercise assistant portion (210) comprises an insertion portion (126) inserted into an insertion groove (121) of the support body (120), a protrusion portion (211) connected to the end of the insertion portion (126), and a first wing body (213) and a second wing body (214) arranged at both sides of the protrusion portion (211). The insertion portion (126) is described above.

The protrusion portion (211) protrudes in a direction of crossing the length direction of the tooth engagement portion (115) by being coupled to the support body (120). The first wing body (213) protrudes in a form curved from one side of the protrusion portion (211). The second wing body (214) protrudes in a form curved from the other side of the protrusion portion (211) so as to achieve symmetry with the first wing body (213) so that the protrusion portion (211) can be disposed therebetween.

As shown in FIG. 9(a), the user using the tongue exercise apparatus (200) according to the present embodiment moves the exercise assistant portion (210) forward with the tongue (10) in a state of inserting the exercise assistant portion (210) in the mouth, thereby training the tongue muscles. And, as shown in FIG. 9(b), the user may train the tongue muscles by putting the tongue (10) on the left or right side of the exercise assistant portion (210) and pushing in the opposite direction repeatedly.

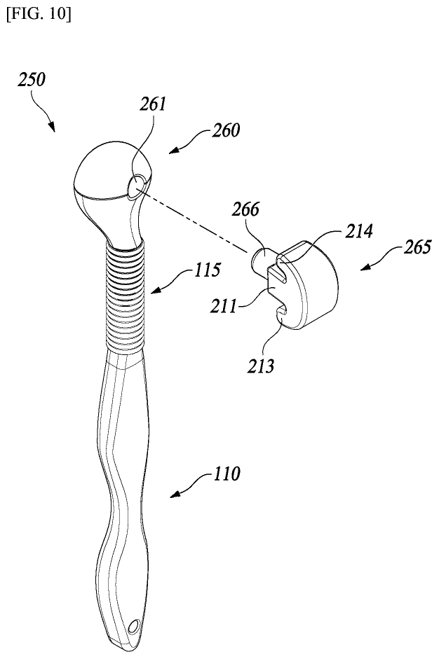

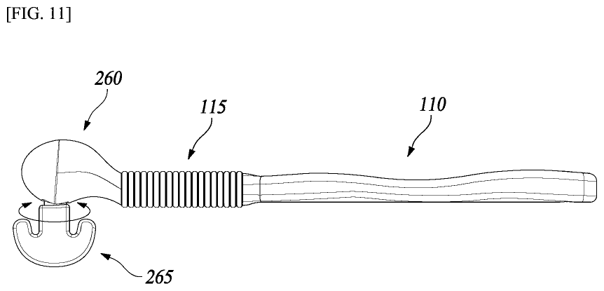

A tongue exercise apparatus (250) shown in FIGS. 10 and 11 includes a handle (110), a tooth engagement portion (115) connected to the end of the handle (110), a support body (260) connected to the end of the tooth engagement portion (115), and an exercise assistant portion (265) coupled to the support body (260). In this tongue exercise apparatus (250), the support body (260) and the exercise assistant portion (265) are modified compared to the tongue exercise apparatus (200) described above.

That is, the support body (260) may rotatably support the exercise assistant portion (265). An insertion groove (261) into which the exercise assistant portion (265) is inserted is provided in the support body (260). The insertion groove (261) is made of a circular cylinder shape. The exercise assistant portion (265) includes an insertion portion (266) inserted into the insertion groove (261), a protrusion portion (211) connected to the insertion portion (266), and a first wing body (213) and a second wing body (214) disposed on both sides of the protrusion portion (211). The insertion portion (266) is made of a circular cylinder shape corresponding to the insertion groove (261).

As shown in FIG. 11, the exercise assistant portion (265) may rotate 360 degrees while being coupled to the support body (260).

Therefore, a user using the tongue exercise apparatus (250) according to the present embodiment can train tongue muscles by pushing the exercise assistant portion (265) with the tongue (10) in a state in which the exercise assistant portion (265) is placed in the mouth. And, the user can train the tongue muscles by pushing the first wing body (213) or the second wing body (214) with the tongue (10) and rotating the exercise assistant portion (265).

In the tongue exercise apparatus (250) according to the present embodiment, since the exercise assistant portion (265) can exhibit more various movements, the exercise assistant portion (265) can assist the tongue 10 to perform more various exercises, and effectively train the tongue muscles.

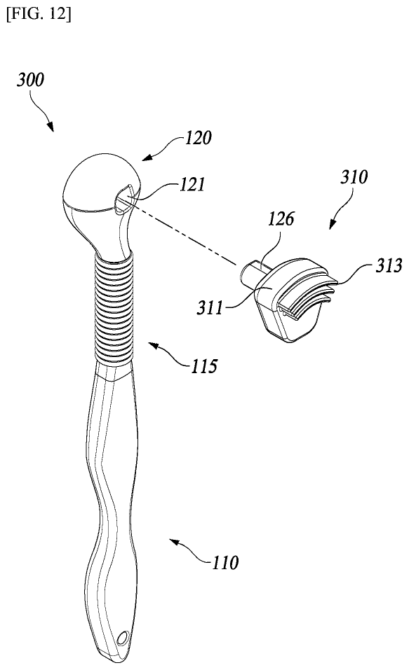

A tongue exercise apparatus (300) shown in FIG. 12 includes a handle (110), a tooth engagement portion (115) connected to the end of the handle (110), a support body 120 connected to the end of the tooth engagement portion (115), and an exercise assistant portion (310) coupled to the support body (120). In this tongue exercise apparatus (300), the exercise assistant portion (310) is modified compared to the tongue exercise apparatus (100) described above.

The exercise assistant portion (310) is detachably coupled to the support body (120) so as to be inserted into the inside of the user's mouth. The exercise assistant portion (310) includes an insertion portion (126) inserted into the insertion groove (121) of the support body (120), a protrusion portion (311) connected to the end of the insertion portion (126), and a plurality of scrapers (313) protruding from the protrusion portion (311). The insertion portion (126) is described above.

The protrusion portion (311) protrudes in a direction of crossing the length direction of the tooth engagement portion (115) by being coupled to the support body (120). The protrusion portion (311) has a shape in which a width gradually decreases from one end toward the other end. That is, the width of one end of the protrusion portion (311) facing the inside of the mouth is greater than the width of the other end of the protrusion portion (311) facing the outside of the mouth.

The plurality of scrapers (313) are disposed on one surface of the protrusion portion (311) to be spaced apart from each other in a direction from one end to the other end of the protrusion portion (311). The scraper (313) may come into contact with the user's tongue 10 and scrape foreign substances attached to the user's tongue (10). The scraper (313) may have a bent plate-like structure. That is, the scraper (313) is formed in an arcuate bent shape so that the central portion of the scraper (313) can be located deeper in the user's mouth than both edges of the scraper (313). In addition, among the plurality of scrapers (313), the length of one or more scrapers (313) disposed relatively closer to one end of the protrusion portion (311) is longer than the length of another or other scrapers (313) disposed relatively closer to the other end of the protrusion (311).

As shown in FIG. 14(a), the user using the tongue exercise apparatus (300) according to the present embodiment moves the exercise assistant portion (310) forward with the tongue (10) while putting the exercise assistant portion (310) in the mouth to train the tongue muscles. In addition, as shown in FIG. 14(b), the user can remove foreign substances such as coat stuck on the tongue (10) by putting the upper portion of the tongue (10) on the scraper (313) and reciprocating it. Therefore, the tongue (10) can be washed and cleaned, and through this, bad breath can be removed.

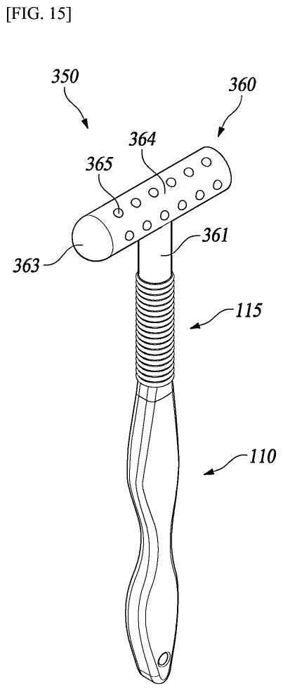

The tongue exercise apparatus (350) illustrated in FIG. 15 includes a handle (110), a tooth engagement portion (115) connected to the end of the handle (110), and an exercise assistant portion (360) connected to the end of the tooth engagement portion (115). Here, the handle (110) and the tooth engagement portion (115) are as described above.

The exercise assistant portion (360) includes a connection portion (361) connected to the end of the tooth engagement portion (115), and a protrusion portion (363) connected to the end of the connection portion (361). The protrusion portion (363) is disposed substantially perpendicular to the connection portion (361) at an end of the connection portion (361). The protrusion portion (363) is provided with a base surface portion (364), and a plurality of protrusions (365) protrudes from the base surface portion (364). The base surface portion (364) may be formed in a curved shape or a flat shape as illustrated. The exercise assistant portion (360) may be made of the same material as the tooth engagement portion (115) and manufactured to be integrated with the tooth engagement portion (115).

The user using the tongue exercise apparatus (350) according to the present embodiment repeats an operation of pushing the central part, the left part or the right part of the exercise assistant portion (360) with the tongue (10) while putting the exercise assistant portion (360) in the mouth to train your tongue muscles. In addition, the user can remove foreign substances such as cover stuck on the tongue (10) through the plurality of protrusions (365) by placing the upper part of the tongue (10) against the base surface portion (364) and reciprocating it.

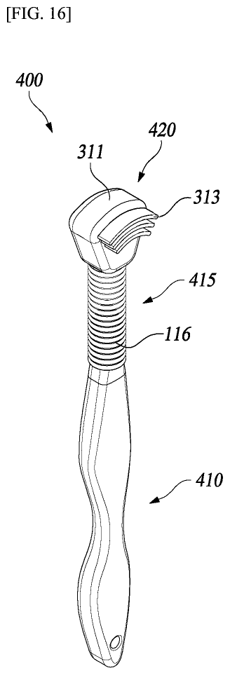

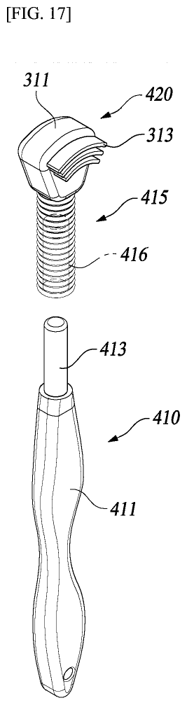

A tongue exercise apparatus (400) illustrated in FIGS. 16 and 17 includes a handle (410), a tooth engagement portion (415) connected to the end of the handle (410), and an exercise assistant portion (420) connected to the end of the tooth engagement portion (415).

The handle (410) includes a handle body (411) which a user can hold by hand, and a coupling protrusion (413) protruding from the end portion of the handle body (411).

The tooth engagement portion (415) is detachably coupled to the handle (410). The tooth engagement portion (415) is provided with a plurality of engagement grooves (116) into which the ends of the user's upper teeth 20 can be inserted. A handle coupling groove (416) corresponding to the coupling protrusion (413) of the handle (410) is provided at one end of the tooth engagement portion (415). The tooth engagement portion (415) is detachably coupled to the handle (410) in such a way that the coupling protrusion (413) is inserted into the handle coupling groove (416).

The exercise assistant portion (420) includes a protrusion portion (311) coupled to the end of the tooth engagement portion (415), and a plurality of scrapers (313) protruding from the protrusion portion (311). The protrusion portion (311) and the scrapers (313) are described above. The exercise assistant portion (420) may be made of the same material as the tooth engagement portion (415) and manufactured to be integrated with the tooth engagement portion (415).

In this tongue exercise apparatus (400), the tooth engagement portion (415) and the exercise assistant portion (420) can be replaced. Therefore, while sharing the handle 410 with other people, only the tooth engagement portion (415) and the exercise assistant portion (420) can be replaced to be used. For example, according to the user's oral structure, tooth structure, etc., tooth engagement portions having various lengths, tooth engagement portions having engagement grooves of various sizes, or tooth engagement portions having engagement grooves of various intervals can be replaced to be used as necessary.

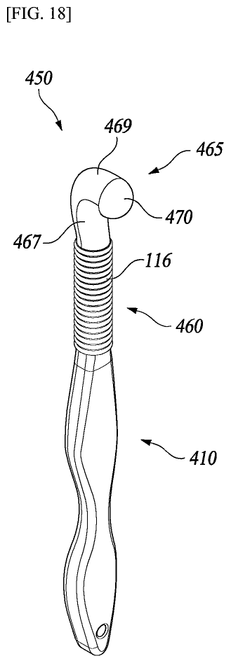

A tongue exercise apparatus 450 shown in FIGS. 18 to 20 includes a handle (410), a tooth engagement portion (460) connected to the end of the handle (410), and an exercise assistant portion (465) connected to the end of the tooth engagement portion (460). The handle (410) includes a handle body (411) and a coupling protrusion (413), as illustrated in FIG. 17.

The tooth engagement portion (460) is detachably coupled to the handle (410). The tooth engagement portion (460) is provided with a plurality of engagement grooves (116) into which the ends of the user's upper teeth (20) can be inserted. A handle coupling groove (416) corresponding to the coupling protrusion (413) of the handle 410 is provided at one end of the tooth engagement portion (460). And, the other end of the tooth engagement portion (460) is provided with an exercise assistant portion coupling groove (461). The tooth engagement portion (460) is detachably coupled to the handle (410) in such a way that the coupling protrusion (413) is inserted into the handle coupling groove (416).

An exercise assistant portion (465) includes an insertion protrusion (466) corresponding to the exercise assistant portion coupling groove (461) of the tooth engagement portion (460), a connection portion (467) connected to the end of the insertion protrusion (466), and a protrusion portion (469) connected to the connection portion (467). The exercise assistant portion (465) may be detachably coupled to the tooth engagement portion (460) in such a way that the insertion protrusion (466) is inserted into the motion auxiliary unit coupling groove 461 or separated from the motion auxiliary unit portion coupling groove (461). The protrusion portion (469) has a bent shape at the connecting portion (467) so as to protrude in a direction of crossing the length direction of the tooth engagement portion (460). A semicircular spherical portion (470) is provided at the end of the protrusion portion (469).

The user using the tongue exercise apparatus (450) according to the present embodiment inserts the exercise assistant portion (465) in the mouth and puts the tongue (10) on the protrusion portion (469) and pushes forward, or puts the tongue 10 on the spherical portion (470) and pushes upwards to train your tongue muscles.

The tongue exercise apparatus (450) according to the present embodiment can be used by replacing the tooth engagement part (460) or the exercise assistant portion (465) while the handle 410 is shared with other people.

FIG. 21 illustrates various modified examples of an exercise assistant portion.

An exercise assistant portion (480) shown in FIG. 21(a) has the same outer appearance as the exercise assistant portion (125) shown in FIG. 2, and a reinforcing material or stiffener (481) is disposed in the exercise assistant portion (480). The reinforcing material or stiffener (481) may be made of a plate spring or other various materials having elasticity and capable of increasing the elastic restoring force of the exercise assistant portion (480).

An exercise assistant portion (485) shown in FIG. 21(b) has the same outer appearance as the exercise assistant portion (210) illustrated in FIG. 7, and has an elastically deformable reinforcing material or stiffener (486) therein. By having the reinforcing material or stiffener (486) inside the exercise assistant portion (485), the exercise assistant portion (485) can increase elastic restoring force and have excellent durability.

An exercise assistant portion (490) shown in FIG. 21(c) has the same outer appearance as the exercise assistant portion (490) illustrated in FIG. 18, and an elastically deformable reinforcing material or stiffener (491) is disposed in the exercise assistant portion (490).

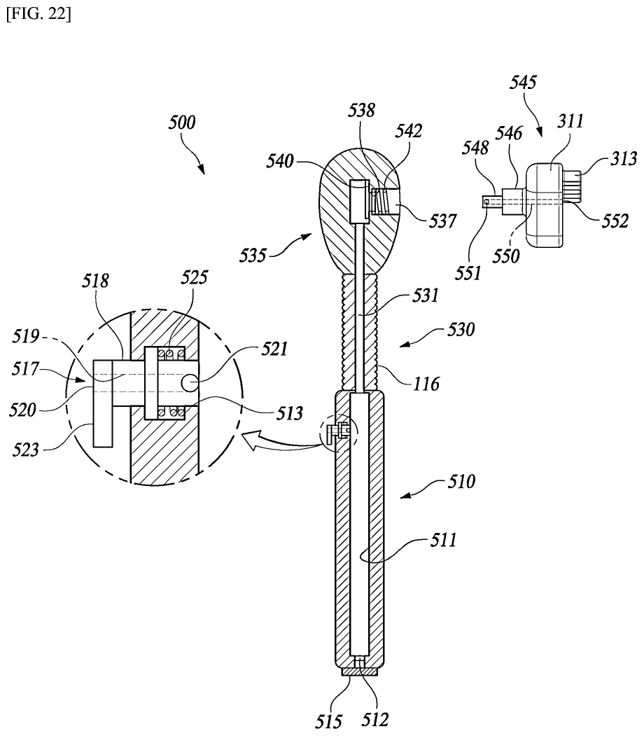

A tongue exercise apparatus (500) illustrated in FIGS. 22 to 24 includes a handle (510), a tooth engagement portion (530) connected to the end of the handle (510), a support body (535) connected to the end of the tooth engagement portion (530), and an exercise assistant portion (545) coupled to the support body (535). The tongue exercise apparatus (500) may contain a liquid such as a mouthwash, gargle or the like, and can supply the liquid into the user's mouth through the exercise assistant portion (545).

The handle (510) has a bar shape that a user can hold with one hand. A handle chamber (511) capable of accommodating liquid is provided inside the handle (510). An inlet hole (512) for injecting liquid into the handle chamber (511) is formed at one side of the handle (510). In addition, an air hole (513) through which external air can be provided into the handle chamber (511) is formed at the other side of the handle (510). The inlet hole (512) may be closed or sealed by a cap (515) coupled to the handle (510).

A valve (517) is installed at the air hole (513). The valve (517) regulates the flow of air through the air hole (513). The valve (517) includes a plunger (518) movably installed at the air hole (513) and a spring (525) that elastically supports the plunger (518). The spring (525) is disposed in the air hole (513) and applies an elastic force to the plunger (518) in a direction away from the handle chamber (511).

A guide passage (519), an inlet hole (520), and an outlet hole (521) are provided at the plunger (518). The inlet hole (520) is provided at one side of the plunger (518) to be exposed to the outside, and the outlet hole (521) is provided at the other side of the plunger (518) to be placed in the handle chamber (511). The guide passage (519) connects the inlet hole (520) and the outlet hole (521). A lever 523 for user's manipulation is provided at an end of the plunger (518).

If no other external force is applied to the plunger (518), the valve (517) maintains a state in which the plunger (518) is pushed away from the handle chamber (511) by the elastic force of the spring (525). At this time, the outlet hole (521) of the plunger (518) moves away from the handle chamber (511) and is located in the air hole (513), thereby blocking air flow through the air hole (513). Therefore, air cannot be flowed into the inside of the handle chamber (511) through the air hole (513).

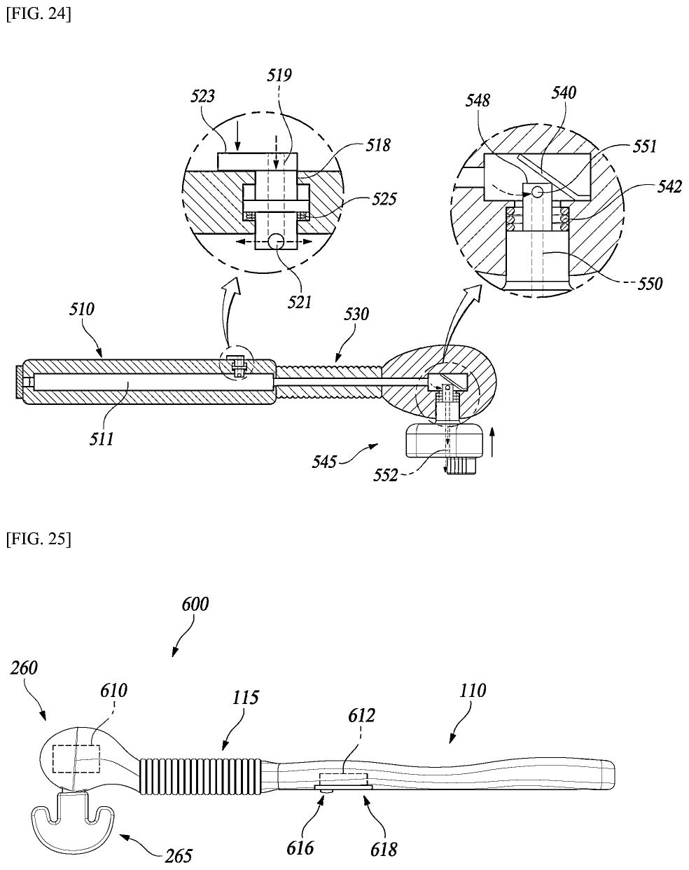

On the other hand, as illustrated in FIG. 24, when the user presses the plunger (518), the outlet hole (521) is placed in the handle chamber (511). At this time, external air may flow along the guide passage (519) and be flowed into the handle chamber (511) through the outlet hole (521).

The tooth engagement portion (530) extends from the end of the handle (510). The tooth engagement portion (530) is provided with a plurality of engagement grooves (116) into which the ends of the user's upper teeth (20) can be inserted. And, the tooth engagement portion chamber (531) connected to the handle chamber (511) is provided inside the tooth engagement portion (530) so that liquid can be accommodated therein.

The support body (535) is connected to the end of the tooth engagement portion (530) to support the exercise assistant portion (545). A support body chamber (536) connected to the tooth engagement chamber (531) is provided inside the support body (535) so that liquid can be accommodated therein. In addition, an insertion groove (537) into which the exercise assistant portion (545) is inserted is provided at one side of the support body (535). And, a connection hole (538) connecting the support body chamber (536) and the insertion groove (537) is provided inside the support body (535). An opening and closing structure (540) capable of opening and closing the connection hole (538) is installed inside the support body chamber (536). The opening and closing structure (540) may be elastically deformed. The opening and closing structure (540) may close and seal the connection hole (538) when there is no external force, and may open the connection hole (538) by being elastically deformed in response to an external force. A spring (542) is installed in the insertion groove (537). The spring (542) may push the exercise assistant portion (545) inserted into the insertion groove (537) in an outward direction of the insertion groove (537).

The exercise assistant portion (545) is coupled to the support body (535) so as to be capable of being inserted into the inside of the user's mouth. The exercise assistant portion (545) includes an insertion portion (546) inserted into the insertion groove (537) of the support body (535), a protrusion portion (311) connected to an end of the insertion portion (546), and a plurality of scrapers (313) protruding from one side of the protrusion portion (311), and an operation protrusion (548) protruding in a direction opposite to a protruding direction of the scraper (313) at the other side of the protrusion portion (311). The protrusion portion (311) and the scraper (313) are as shown in FIG. 12. The operation protrusion (548) may protrude from the insertion portion (546) to contact the opening and closing structure (540) and press the opening and closing structure (540) in the direction of opening the connection hole (538).

A passage (550) is provided inside the exercise assistant portion (545). The passage (550) extends from the operation protrusion (548) to the outer surface of the protrusion portion (311). Accordingly, the passage (550) connects an opening (551) formed at the operation protrusion (548) and an outlet hole (552) formed on the outer surface of the protrusion portion (311). Therefore, when the operation protrusion (548) enters into the inside of the support chamber (536), the liquid in the support chamber (536) can flow along the opening (551) and the passage (550) and can be discharged to the outside the protrusion portion (311) through the outlet hole (552).

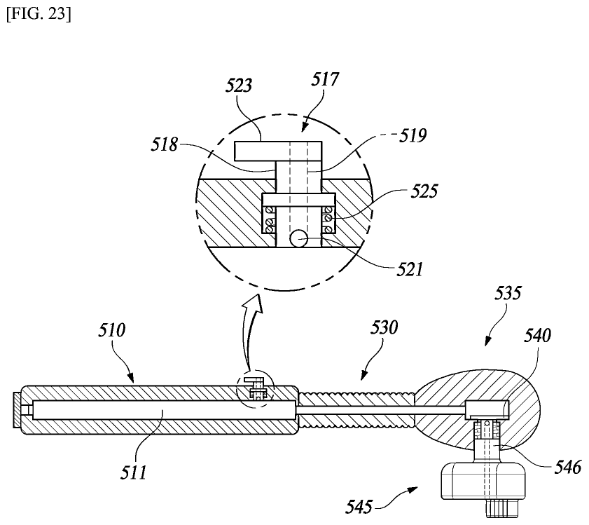

As illustrated in FIG. 23, a tongue exercise apparatus (500) according to the present embodiment may couple the exercise assistant portion (545) to the support body (535), put the exercise assistant portion (545) in the mouth and be used to perform tongue exercise.

Therefore, the user using the tongue exercise apparatus (500) according to the present embodiment inserts the exercise assistant portion (545) into the mouth, puts the upper teeth (20) on the tooth engagement portion (530), and pushes the exercise assistant portion (545) with the tongue (10) while fixing the position of the exercise assistant portion (545) to train the tongue muscles.

Additionally, the user may push the exercise assistant portion (545) toward the support body (535) with the tongue (10) to receive the liquid in the mouth. Accordingly, as shown in FIG. 24, when the spring (542) is compressed by pressing the exercise assistant portion (545) toward the support body (535) with the tongue (10), the operation protrusion (548) pushes the opening and closing structure (540) and then is entered into the inside of the support chamber (536). At this time, because the opening (551) of the operation protrusion (548) is located in the support chamber (536), the liquid in the support chamber (536) flows along the opening (551) and the passage (550) and is discharged through the outlet hole (552). Therefore, the liquid can be supplied to the user's tongue (10) and the inside of the mouth. As described above, the user can press the plunger (518) to allow external air to flow into the handle chamber (511) through the air hole (513) so that the liquid can smoothly flow into the exercise assistant portion (545).

The user is supplied with the liquid such as mouthwash or gargle, and puts the tongue (10) on the scraper (313) and rubbing the tongue (10), thereby effectively removing foreign substances attached to the tongue (10) and effectively removing bad breath.

Since the exercise assistant portion (545) can move the opening and closing structure (540) only when a pressure greater than a certain level is applied, the user can push the exercise assistant portion (545) toward the support body (535) only when necessary to receive liquid supply.

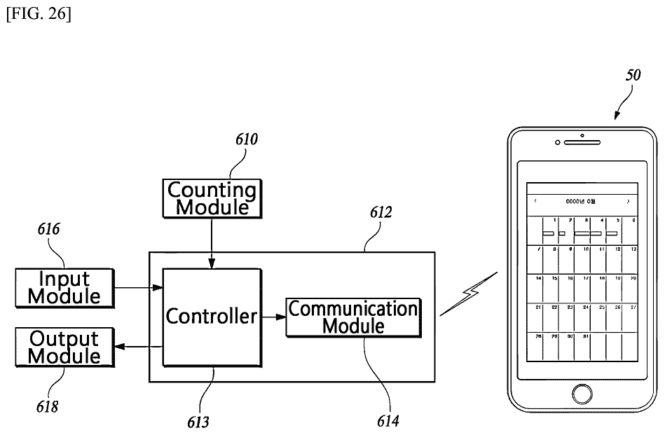

A tongue exercise apparatus (600) shown in FIGS. 25 and 26 includes a handle (110), a tooth engagement portion (115) connected to the end of the handle (110), a support body (260) connected to the end of the tooth engagement portion (115), an exercise assistant portion (265) coupled to the support body (260), a counting module (610) configured to count the number of times the motion assistant portion (265) presses or moves, a control module (612) configured to receive measurement information from the counting module (610), an input module (616) and an output module (618). In the tongue exercise apparatus (600) according to the present embodiment, components such as the counting module (610) and the control module (612) in the tongue exercise apparatus (250) as shown in FIG. 10 are installed.

The counting module (610) is installed at the support body (260) to be adjacent to the exercise assistant portion (265) and counts the number of times the exercise assistant portion (265) is pressed or moved by the user's tongue (10). Because the exercise assistant portion (265) is rotatably installed at the support body (260), the counting module (610) can count the number of rotations rotated by pushing the exercise assistant portion (265) with the user's tongue (10). Here, the rotation angle of the exercise assistant portion (265) counted by the counting module (610) may be set in various ways. For example, the counting module (610) may be set to count the number of times the exercise assistant portion (265) rotates by 360 degrees, or may be set to count the number of rotations of the exercise assistant portion (265) by 180 degrees.

Although the figure shows that the counting module (610) is installed at the support body (260), the installation position of the counting module (610) may be variously changed.

The control module (612) includes a controller (613) and a communication module (614). The controller (613) receives measurement information from the counting module (610), receives an input signal from the input module (616), and controls the output module (618) and the communication module (614). The controller (613) may receive measurement information from the counting module (610) and output the measurement information through the output module (618). And, the controller (613) may receive measurement information and transmit the measurement information to the personal terminal (50) of the user through the communication module (614). The communication module (614) may transmit information from the tongue exercise apparatus (600) to the personal terminal (50) or receive information transmitted from the personal terminal (50).

The input module (616) may be installed at the handle (110) for user manipulation. The input module (616) may be formed of various structures capable of generating input signals according to the user manipulations such as an input button or a touch screen. According to the manipulation of the input module (616), information may be output to the output module (618) or measurement information may be transmitted to the personal terminal (50), and information may be received from the personal terminal (50) and output to the output module (618).

The output module (618) is installed on one side of the outer surface of the handle (110) to provide various information about the use of the tongue exercise apparatus (600) to the user. The output module 618 may be implemented by various display panels.

The user of the tongue exercise apparatus (600) according to the present embodiment installs an application related to the use of the tongue exercise apparatus (600) in the user's personal terminal (50) to store and manage measurement information received from the tongue exercise apparatus (600). For example, the application related to the tongue exercise apparatus (600) may provide a date when the user performed a tongue exercise using the tongue exercise apparatus (600), an exercise time for each date, the number of exercises, and an exercise intensity. In this case, the user can check whether snoring is improved according to the tongue exercise by viewing the exercise records for each day.

In this way, the tongue exercise apparatus (600) is used in conjunction with the user's personal terminal (50), thereby increasing convenience of use and providing motivation of the exercise to the user.

Although not shown in the drawings, a battery or power supply terminal for power supply may be installed at the handle (110).

In the present embodiment, the exercise assistant portion may be changed to an exercise assistant portion having various structures described above, in addition to the rotary type. In this case, the counting module (610) can be set to count the number of time the exercise assistant portion is pressed by the user's tongue in various ways by counting the number of times the exercise assistant portion is pushed and deformed by the user's tongue (10), or counting the number of times a pressure of a certain size or more is applied to the exercise assistant portion.

Although the preferred examples of the present disclosure have been described above, the scope of the present invention is not limited to the forms described and illustrated above.

For example, although it has been described above that the user's upper teeth are inserted into the engagement grooves, the tongue exercise apparatus according to the present disclosure can be used such that the user's lower teeth are inserted into the engagement grooves.

In the above, the present disclosure has been shown and described in relation to preferred embodiments for illustrating the principles of the present invention, but the present invention is not limited to the configurations and operations as shown and described. Rather, it would be appreciated by those skilled in the art that various changes and modifications may be made to the present invention without departing from the spirit and scope of the appended claims.

What is claimed is:

1. A tongue exercise apparatus which a user is able to hold with a hand and use to exercise a tongue, the tongue exercise apparatus comprising:
a handle which the user is able to hold with the hand;
a teeth engagement portion extended from an end portion of the handle, and having one or more engagement grooves configured to be engageable with an end portion of teeth of the user; and
an exercise assistant portion connected to the teeth engagement portion to be insertable into an inside of a mouth of the user, and having a protrusion portion protruding in a direction of crossing with a direction in which the teeth engagement portion is extended from the handle to be contactable with the tongue of the user.

2. The tongue exercise apparatus according to claim 1, wherein a plurality of the engagement grooves are disposed to be spaced apart from each other along the direction in which the teeth engagement portion is extended from the handle.

3. The tongue exercise apparatus according to claim 1, wherein the teeth engagement portion is made of a circular cylinder shape, and the one or more engagement grooves are provided in an annular shape on an outer circumferential surface of the teeth engagement portion.

4. The tongue exercise apparatus according to claim 1, wherein the exercise assistant portion is made of silicon material.

5. The tongue exercise apparatus according to claim 1, wherein a reinforcing material or stiffener configured to be elastically deformable is disposed inside the exercise assistant portion.

6. The tongue exercise apparatus according to claim 1, wherein the teeth engagement portion and the exercise assistant portion are made of silicon material and are formed integrally with each other.

7. The tongue exercise apparatus according to claim 6, wherein the teeth engagement portion is replaceably coupled to the handle.

8. The tongue exercise apparatus according to claim 1, further comprising a support body disposed at an end of the teeth engagement portion to support the exercise assistant portion.

9. The tongue exercise apparatus according to claim 8, wherein the exercise assistant portion is replaceably coupled to the support body.

10. The tongue exercise apparatus according to claim 8, wherein the exercise assistant portion is rotatably coupled to the support body.

11. The tongue exercise apparatus according to claim 10, wherein the exercise assistant portion comprises:
a first wing body protruded from one side of the protrusion portion; and
a second wing body protruded from an other side of the protrusion portion.

12. The tongue exercise apparatus according to claim 8, wherein the support body comprises a contact curved surface portion formed in a curved surface shape to be capable of contact a roof of the mouth of the user.

13. The tongue exercise apparatus according to claim 8, further comprising:
a handle chamber disposed inside the handle to store liquid;
a teeth engagement portion chamber disposed inside the teeth engagement portion to be connected to the handle chamber;
a support body chamber disposed inside the support body to be connected to the teeth engagement portion chamber;
an insertion groove formed at one side of the support body;
a connection hole formed at an inside of the support body to connect the support body chamber and the insertion groove;
an opening and closing structure installed at the support body chamber and configured to open and close the connection hole;
a spring installed at the insertion groove to apply an elastic force to the protrusion portion in a direction away from the connection hole;
an insertion portion protruding at one side of the protrusion portion;
an operation protrusion protruding at the insertion portion to be contacted with the opening and closing structure and press the opening and closing structure in a direction of opening the connection hole; and
a passage extended from the operation protrusion to an outer surface of the protrusion portion to guide the liquid of the support body chamber to the outer surface of the protrusion portion.

14. The tongue exercise apparatus according to claim 13, further comprising:
an air hole formed at the handle to flow outside air into the handle chamber;
a plunger movably installed in the air hole to regulate flow of air through the air hole; and
a spring installed inside the air hole to apply an elastic force to the plunger in a direction of blocking the flow of the air through the air hole.

15. The tongue exercise apparatus according to claim 1, wherein the exercise assistant portion comprises:
a curved portion provided on one side of the protrusion portion to be contacted with the tongue of the user; and
an assistant protrusion portion protruding from a center of an end of the protrusion portion to be contactable with the tongue of the user.

16. The tongue exercise apparatus according to claim 1, wherein the exercise assistant portion comprises a scraper protruding from the assistant protrusion portion so that the scraper contacts the tongue of the user to scrape away a foreign substance on the tongue of the user.

17. The tongue exercise apparatus according to claim 1, wherein the scraper is formed of an arcuately bent shape so that a center portion of the scraper is positioned deeper in the mouth of the user than both edges of the scraper.

18. The tongue exercise apparatus according to claim 1, wherein the protrusion portion has a base surface portion,
wherein a plurality of protrusions are protrudingly formed on the base surface portion.

19. The tongue exercise apparatus according to claim 1, further comprising:
a counting module configured to count a number of times the protrusion portion is pressed or moved by the tongue of the user.

20. The tongue exercise apparatus according to claim 19, further comprising:
a controller configured to receive measurement information from the counting module, and transmit the measurement information to a personal terminal of the user.

21. The tongue exercise apparatus according to claim 19, further comprising:
an output module configured to output the measurement information of the counting module.
